(19) United States
(12) Patent Application Publication
(10) Pub. No.: US 2020/0246171 A1
(43) Pub. Date: Aug. 6, 2020

(54) Orthopedic Device for Anterior Cruciate Ligament Complications

(71) Applicant: Ossur Iceland ehf, Reykjavik (IS)

(72) Inventors: Harry Duane ROMO, Foothill Ranch, CA (US); Bjorn OMARSSON, Reykjavik (IS); Sindri Pall SIGURDSSON, Reykjavik (IS)

(21) Appl. No.: 16/783,635

(22) Filed: Feb. 6, 2020

Related U.S. Application Data
(60) Provisional application No. 62/801,937, filed on Feb. 6, 2019.

(51) Int. Cl. A61F 5/01; A61F 5/30

(52) U.S. Cl. CPC A61F 5/0125; A61F 2005/0167; A61F 5/30

(57) ABSTRACT

An orthopedic device for treatment of anterior cruciate ligament complications, including a knee support with a strap system for providing variable assistance during gait, particularly as a user flexes a knee. A method for using the orthopedic device involves providing increased loading at certain flexion angles and diminished loading at other flexion angles and extension.

### FIELD OF THE DISCLOSURE

This disclosure relates to an orthopedic device for treatment of anterior cruciate ligament complications, and more particularly to a knee support with a strap system for providing variable assistance during gait, particularly as a user flexes a knee. A method involves the orthopedic device by generally providing increased loading at certain flexion angles and diminished loading at other flexion and extension angles.

### BACKGROUND

There are roughly 200,000 partial or complete anterior cruciate ligament (“ACL”) injuries per year in the U.S. The ACL is an intracapsular ligament that cannot spontaneously heal complete tears. Approximately 50% or 100,000 U.S. patients of ACL injuries annually go untreated either through a lack of diagnosis or because repair is deemed unnecessary due to the patient's low level of activity.

Partial ACL tears may heal spontaneously but may heal at an increased length resulting in a positive “Drawer Test,” a commonly used test to detect the rupture of cruciate ligaments in the knee and the ability of the tibia to shift anteriorly regarding the femur. In the Drawer Test, if the tibia pulls forward or backward more than normal, the test is positive. Excessive displacement of the tibia anteriorly indicates the ACL is likely torn, whereas excessive posterior displacement of the tibia indicates the PCL is probably torn. Complete ACL tears that go unrepaired also will cause a positive Drawer Test.

Some surgeons show positive results in healing for some patients with ACL injury through minor surgical intervention by reattaching the ACL at what is known as a graft.

For the remaining 100,000 patients who undergo ACL repair for complete tears, some studies have shown the strength of the ACL ligament is reduced to approximately 50% of its original strength six months after surgery. This may be due to the revascularization of the ACL ligament. At six months, these patients often feel stable enough to return to their previous level of activity. This places the ACL at risk because the patient does not realize the ACL is only at 50% of its original strength.

During normal activity, tension on the ACL may vary. Activities that require sudden stops and changes of direction may place high tension on the ACL or create displacement of the tibia regarding the femur. Adjustment of the tibia relative to the femur, and possibly reduction of ACL tension, could benefit the patient in the following ways: 1) reduce additional injury and preserve the length of the partially torn ACL, and 2) reduce the risk of reinjury of the graft for the ACL-repaired patient.

Orthopedic braces and supports are arranged to restrict, inhibit, immobilize, or otherwise control motion about anatomical segments or joints of the human body. These braces and supports provide compression, support, and stability. Many known braces and supports incorporate rigid members and hinges for immobilizing a joint or facilitating movement of the joint. Orthopedic bracing has tended toward greater rigidity to provide ultimate immobilization with a reduced margin of error in mobility, thus many device designs are over-engineered.

There tends to be an inverse relationship between the rigidity of the device and patient comfort/compliance. The more rigid the device is, the less likely it will be worn, especially over extended periods of time. If the patient removes a required device to increase comfort, rehabilitation may be hampered and risk of further injury may be elevated.

Flexible braces and supports exist which offer compression, support, and stability. However, many are formed from synthetic materials such as Neoprene. These types of braces can give rise to allergic reactions and can get hot. Because they typically rely on a sheet of homogeneous material, they lack areas having different properties and may be ill-fitting or ineffective as a joint requires different areas of compression.

Orthopedic devices, such as flexible orthopedic devices and sleeves, provide anatomical fit by conforming to a user's anatomy for physiologically correct support. They are flexible and arranged for contouring to a body or joint to minimize movement restriction and discomfort. The devices may be configured to stretch in different ways to enable greater muscle stability. In view of their sizing and fit, these devices not only provide support but also improve circulation and reduce pain and inflammation. Other types of flexible orthopedic devices and sleeves may include Neoprene and other textiles and materials. They may include other tubular configurations or other shapes and configurations as well.

From these observations, there is a need for an orthopedic device that is arranged to treat, prevent or mitigate ACL injuries while offering a flexible brace with the benefits of known ACL braces and without the attendant drawbacks of a rigid brace.

### SUMMARY

According to exemplary orthopedic device for anterior cruciate ligament complications embodiments of the disclosure, an orthopedic device may take the form of a knee support comprising an elasticized sleeve, preferably formed from a three-dimensional knitted or other suitable material. The elasticized sleeve is arranged to exert generally uniform or gradients of compression about a limb while offering a biomechanical range of motion and therapeutic elements integrated into a single panel, or panels that may form the sleeve.

A preferred embodiment of the orthopedic device may take the form of a knee support and is arranged for providing static anteriorly and/or posteriorly directed forces on the proximal tibia and/or distal femur to provide a physiologically corrective orientation of the knee. The orthopedic device preferably includes a sleeve having a tubular shape, and a strapping arrangement composed of staggered anterior and posterior straps adapted to generate anteriorly and posteriorly directed forces about a knee. The orthopedic device has stays or hinges, preferably on medial and lateral sides of the sleeve upon which the straps are correspondingly anchored and/or adjustably secured.

In another embodiment, the orthopedic device may take the form of a knee support preferably arranged for providing dynamic anteriorly and/or posteriorly directed forces on the proximal tibia and/or distal femur to provide a physiologically corrective orientation of the knee. The orthopedic device preferably includes a sleeve having a tubular shape, and a strapping arrangement composed of anterior and posterior straps and helically extending straps adapted to generate anteriorly and posteriorly directed forces about a knee. The helically extending straps preferably intersect at posterior and anterior locations, proximal and distal of the knee, respectively, and individually attach to the same respective side of the sleeve. The orthopedic device has stays or hinges, preferably on medial and lateral sides of the sleeve upon which the straps are correspondingly anchored and/or adjustably secured.

With the embodiments, loading on the leg, whether of the femur or tibia and on either posterior or anterior sides, is generally achieved at increased amounts depending on and within predetermined angles and of diminished amounts outside the predetermined angles. The reduced loading outside the predetermined angles is preferably gradual and is achieved by means interacting with a hinge articulating from extension to flexion and vice versa.

The strapping systems can be arranged posteriorly above the knee so it is arranged on the distal posterior thigh of a user so as not to impinge on a user's popliteal. In this embodiment, posterior dynamic compression from the strapping system urges the anterior femur relative to the tibia similar in the manner the tibial shell urges the posterior relative to the femur and produces the same dynamic load to assist users with impaired knee ligaments.

A muscle activator may be provided with any of the embodiments to target compression to predetermined muscles, for example, the hamstring muscles. The muscle activator is provided to increase the proprioceptive effect of the muscle activator on muscles, which is intended to assist the muscles to be more active and quicker to respond in physical movements such as jumping and cutting. Regarding the hamstrings, because it is desired to stabilize the knee in the same direction as the ACL, an increase in hamstring activity may make up for a deficiency of the ACL.

The numerous other advantages, features, and functions of embodiments of an orthopedic device will become readily apparent and better understood in view of the following description and accompanying drawings.

### BRIEF DESCRIPTION OF THE DRAWINGS

The drawing figures are not necessarily drawn to scale, but instead are drawn to provide a better understanding of the components thereof, and are not intended to be limiting in scope, but to provide exemplary illustrations. The figures illustrate exemplary configurations of an orthopedic device, and in no way limit the structures or configurations of a liner according to the present disclosure.

FIG. 1 is a perspective view showing a first side of an orthopedic device as a knee support arranged with a static strapping system.

FIG. 2 is a perspective view showing a second side of the knee support of FIG. 1.

FIG. 3 is a front elevational view showing the knee support of FIG. 1.

FIG. 4 is a rear elevational view showing the knee support of FIG. 1.

FIG. 5 is a schematic view of a force diagram of the first side of the knee support of FIG. 1.

FIG. 6 is an elevational view of an exemplary hinge or stay in the knee support of FIG. 1.

FIG. 7 is a perspective view of a muscle activator for use in the knee support of FIG. 1.

FIG. 8 is an elevational view of the muscle activator in the knee support of FIG. 7.

FIG. 9 is an exemplary view showing the muscle activator of FIG. 7 inserting into a knee support of FIG. 1.

FIG. 10 is a schematic view showing the muscle activator of FIG. 7 on a posterior leg.

FIG. 11 is a perspective view showing a first side of another embodiment of an orthopedic device in the form of a knee support.

FIG. 12 is a perspective view showing a second side of the knee support of FIG. 11.

FIG. 13 is an elevational view of the knee support of FIG. 11.

FIG. 14 is a rear elevational view of the knee support of FIG. 12.

FIG. 15 is a schematic view of a force diagram of the first side of the knee support of FIG. 11.

### DETAILED DESCRIPTION OF VARIOUS EMBODIMENTS

### A. Overview

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that, unless a term is defined to possess a described meaning, there is no intent to limit the meaning of such term, either expressly or indirectly, beyond its plain or ordinary meaning.

### B. Definitions

For ease of understanding the disclosed embodiments of an orthopedic device, the anterior and posterior portions of the orthopedic device may be described independently. Anterior and posterior portions of the orthopedic device function together to support and stabilize anatomical portions of the user of the device.

For further ease of understanding the embodiments of an orthopedic device as disclosed, a description of a few terms, when used, is necessary. As used, the term “proximal” has its ordinary meaning and refers to a location next to or near the point of attachment or origin or a central point, or located toward the center of the body. Likewise, the term “distal” has its ordinary meaning and refers to a location situated away from the point of attachment or origin or a central point, or located away from the center of the body.

Medial refers to being toward the midline of the body or the median or mid-sagittal plane, which splits the body, head-to-toe, into two halves, the left and right. Lateral is the side of the body or part of the body that is away from the middle. In the context of the knee, the medial side is on the inside of the knee joint and the lateral side is on the outside of the joint relative to the median plane.

The term “posterior” also has its ordinary meaning and refers to a location behind or to the rear of another location. The term “anterior” has its ordinary meaning and refers to a location ahead of or to the front of another location.

### C. Various Embodiments of the Orthopedic Device

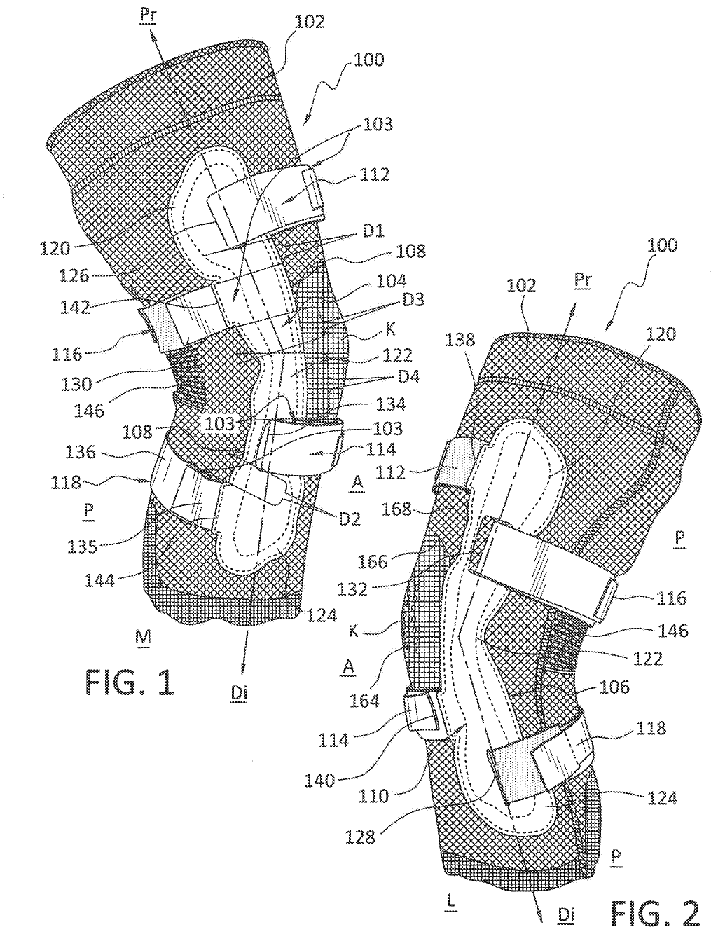

According to an embodiment illustrated in FIGS. 1 and 2, the orthopedic device is a knee support 100 including a sleeve 102 and a strapping system 103. The knee support 100 is arranged to secure onto a user's leg below and above the knee, respectively. The strapping system 103 is arranged to neutralize undesired forces on a patient's ACL in the anterior and posterior directions.

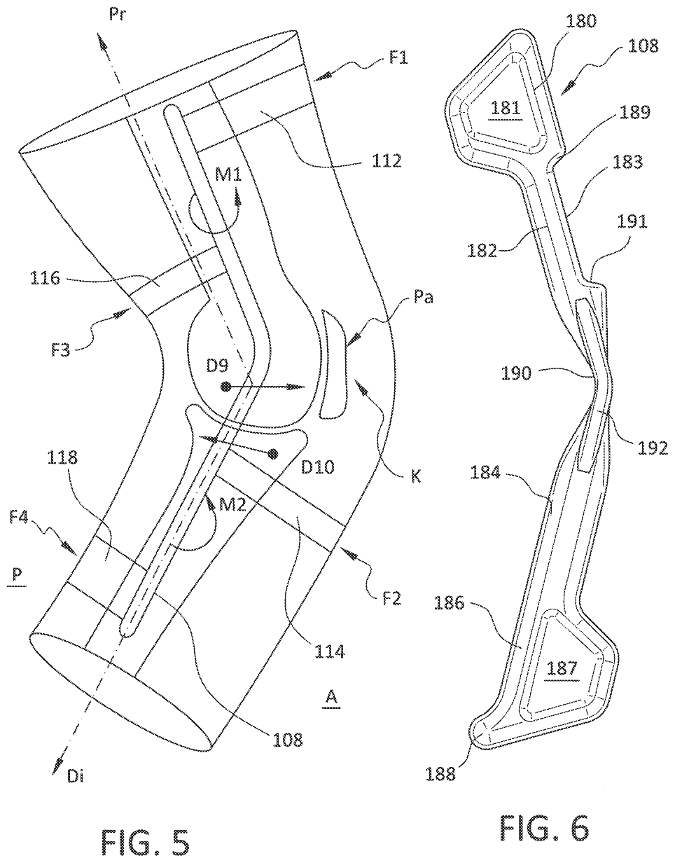

As will be explained in the context of FIG. 5, the knee support 100 and accompanying strapping system 103 provides two three-point loading schemes wherein a first three-point loading scheme includes forces F1, F2 directed to the anterior side of the leg proximal and distal the knee, respectively, to counteract a force F3 directed to the posterior side of the leg distal of the knee. A second three-point loading scheme includes forces F3, F4 directed to the posterior side of the leg proximal and distal of the knee to counteract a force F2 directed to the anterior leg distal of the knee, to maintain an anatomically correct relationship of the femur and tibia.

According to an embodiment, the sleeve 102 generally applies uniform retentive forces and mild compressive forces along its length against a user's leg. The sleeve 102 is preferably formed of an elasticized sleeve such as from a three-dimensional knitted or other suitable material. The elasticized sleeve 102 is arranged to exert generally uniform or gradients of compression about a limb, while offering a biomechanical range of motion and therapeutic elements integrated into a single panel, or panels that may form the sleeve 102. The sleeve 102 is preferably continuously formed along its length between proximal and distal ends of the sleeve 102 and forms a tubular structure.

Alternatively, the knee support 100 may have a rigid or semi-rigid frame including the lower and upper components preferably but not limited to being connected to one another by the hinge assembly on both the medial and lateral sides of the brace. The knee support frame may take on many shapes, such as those shown and described in U.S. Pat. No. 5,230,697, granted Jul. 27, 1993; U.S. Pat. No. 8,048,013, granted on Nov. 1, 2011; U.S. Pat. No. 8,740,829, granted on Jun. 3, 2014; and U.S. Pat. No. 10,413,437, granted on Sep. 17, 2019; each reference being incorporated by reference in its entirety.

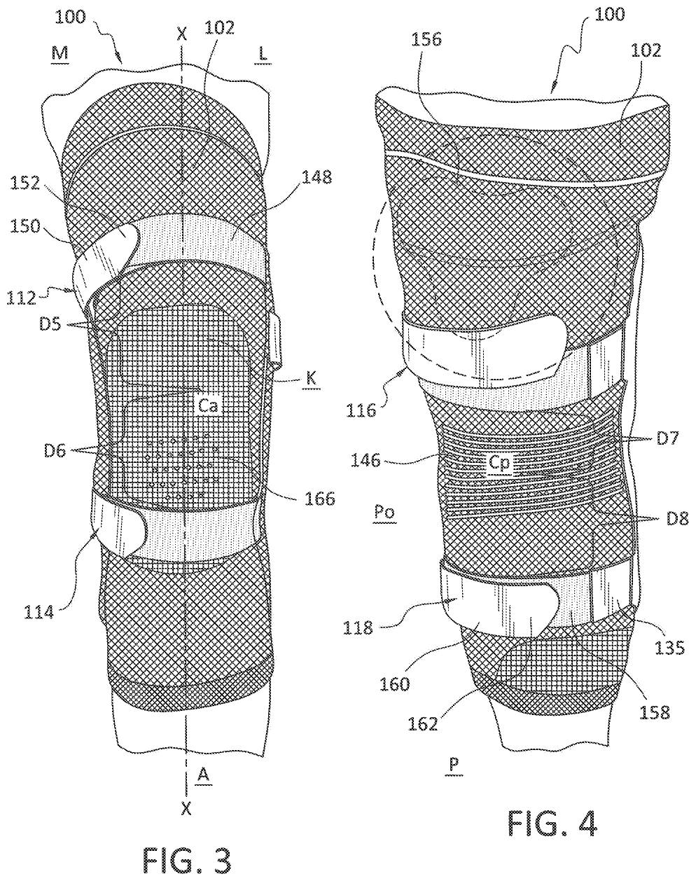

In the embodiment of FIGS. 1-4, the orthopedic device 100 generally comprises the sleeve 102 having a tubular form defined along an axis X-X. The strapping system 103 has a first anterior strap 112 connected to the sleeve 102 and extending between lateral L and medial M sides of the sleeve 102 on an anterior side A thereof; a second anterior strap 114 connected to the sleeve 102 and extending between lateral L and medial M sides of the sleeve 102 on posterior side P thereof; a first posterior strap 116 connected to the sleeve and extending between lateral L and medial M sides of the sleeve 102 on an anterior side A; and a second posterior strap 118 connected to the sleeve and extending between lateral L and medial M sides of the sleeve 102 on the posterior side P.

The first anterior strap 112 and the first posterior strap 116 are located above an anterior center Ca of the sleeve 102. The second anterior strap 114 and the second posterior strap 118 are located below an anterior center Ca of the sleeve 102.

For example, the first posterior strap 116 is located a distance D3 from the anterior center Ca of the sleeve 102. The first anterior strap 112 is located a distance D1 from the first posterior strap 116, and the first posterior strap 116 is located closer to the anterior center Ca than the first anterior strap 112. The relative distance of D3 relative to D1 is determined on the extent that leverage is created by the straps 112, 116 relative to the anterior or posterior knee center Ca, Cp according to the arrangement of the first anterior and posterior straps 112, 116, which contribute to the three-point loading of the femur and tibia.

The second anterior strap 114 is located a distance D4 from the anterior center Ca of the sleeve 102. The second posterior strap 118 is located a distance D2 from the second anterior strap 114. In contrast with the first anterior and posterior straps 112, 116, the second posterior strap 118 located farther from the anterior center Ca than the second anterior strap 114. Like the first anterior and posterior straps 112, 116, their position is determined according to the leverage they form relative to the anterior or posterior center Ca, Cp.

The third and fourth distances D3, D4 of the first and second anterior straps 112, 114 on the anterior side A of the sleeve 102 are greater than corresponding fifth and sixth distances D5, D6 of the first and second anterior straps 112, 114, respectively, from the posterior center Cp on the posterior side P of the sleeve 102. The arrangement of the distances D1, D2, D3, D4s create a staggered formation of the strapping system 103 relative to the axis X-X of the sleeve 102, such that the first and second anterior straps 112, 114 and the first and second posterior straps 116, 118 alternate relative to one another.

The strap system 103 is tethered to first and second panels 104, 106 extending along medial and lateral sides of the sleeve 102. The first and second panels 104, 106 are secured to the sleeve and extend generally parallel to the axis X-X. First and second hinges or stays 108, 110 are preferably located between the first and second panels 104, 106, respectively, and the sleeve 102. An example of the hinges or stays is found in U.S. patent application publication no. 2019/0048923, published on Feb. 14, 2019, incorporated herein in its entirety by reference. Another example of a hinge or stay is found in FIG. 6.

According to the illustrated embodiment, the first panel 104, which holds the same for the second panel 106, includes a first portion 120 located above the anterior center Ca of the sleeve 102, a second portion 124 located below the anterior center Ca of the sleeve 102, and a middle portion 122 located generally at the anterior center Ca of the sleeve 102. The first panel 104 preferably defines a slot 126 through which the first anterior strap 112 extends to engage, wrap around or otherwise secure to the hinge 108. For example, the first hinge or stay 108 defines a first ring 180 defining an opening 181 through which the first anterior strap 112 extends.

The first posterior strap 116 has a first end 130 secured to the first panel 104. The first end 130 is defined by an elasticized segment 130, an inelastic segment of the first posterior strap 116 secured to the elasticized segment 130, and adjustably connects to the second hinge or stay 110 by extending through a third slot 132 formed by the second panel 106. An interface 142 permanently secures the first end 130 to the first panel 104. The first panel 104 forms a second slot 134 through which the second anterior strap 128 extends. The first hinge or stay 108 defines a first or proximal segment 182 defining a recessed segment 183 formed along an anterior side of the first hinge or stay 108. The recessed segment 183 may be defined by shoulders 189, 191 defined by the first segment 182.

The first hinge or stay 108 defines a middle or articulating segment 190 located between first or proximal and second or distal segments 182, 184. The middle segment 190 has a reinforcing rib 192 formed by the first hinge or stay 108 or provided as an insert attached to the middle segment 190. The first hinge or stay 108 defines a second or distal segment 184 which may include a recessed section 183 about which the second anterior strap 114 extends.

A second panel 106 extends along a medial-lateral plane on a second side L of the sleeve 103. The first and second anterior straps 112, 114 and the first and second posterior straps 116, 118 connect to the second panel 106. A second hinge or stay 110 is located between the second panel 106 and the sleeve 102. The second panel 106 defines a second slot 128 through which the second posterior strap 118 extends. The second hinge or stay 110 defines a second ring 186 defining an opening 187 through which the second posterior strap 118 extends. The second hinge or stay 110 forms a wing portion 188 protruding or obliquely protruding from the second ring 186.

The second posterior strap 118 includes an elasticized portion 135 secured to the first panel 104 along an interface 144, and an inelastic portion 136 adjustably secured to the second panel 106. The interface 144 is a permanent connection to the first panel 104. While the first and second posterior straps 108, 110 may include an elasticized segment, the first and second anterior straps 112, 114 are preferably inelastic. An example of the first and second posterior straps 108, 110 can be found in U.S. Pat. No. 9,364,365, granted on Jun. 14, 2016, and incorporated herein by reference in its entirety.

The first and second anterior straps 112, 114 are permanently secured to the second panel 106 along first and second interfaces 138, 140 which are permanent connections to the second panel 106. The first and second posterior straps 116, 118 are adjustably secured to the second panel 106 via the second hinge or stay 110. The first and second anterior straps 112, 114 are adjustably secured to the first panel 104 such that adjustment of the first and second anterior straps 112, 114 is in a first circumferential direction, and adjustment of the first and second posterior straps 112, 114 is in a second circumferential direction opposite the first circumferential direction.

The first and second anterior straps 112, 114 each define an elongate segment 148 and an overlapping segment 150 having a tab 152 securing to the elongate segment 148. The tab 152 carries hook and loop fastener material and the elongate segment 148 has corresponding hook-and-loop fastener material for engagement with the tab 152. The first and second posterior straps 116, 118 each define an elongate segment 158 and an overlapping segment 160 having a tab 162 securing to the elongate segment 158. The tab 162 carries hook and loop fastener material and the elongate segment 158 has corresponding hook and loop fastener material for engagement with the tab 162. The first and second posterior straps 116, 118 may be spaced from the posterior center Cp by seventh and eighth distances D7, D8.

The sleeve 102 defines a popliteal region 146 on a posterior side thereof and has different flexibility from the regions of the sleeve 102 surrounding the popliteal region 146. The sleeve 102 defines a knee locator section 166 having a differently formed textile pattern from a periphery 168 about the knee locator 166. The knee locator section 166 defines a flexibility feature 164. The flexibility feature 164 is defined by a textile pattern providing greater flexibility than the textile pattern about the periphery 168.

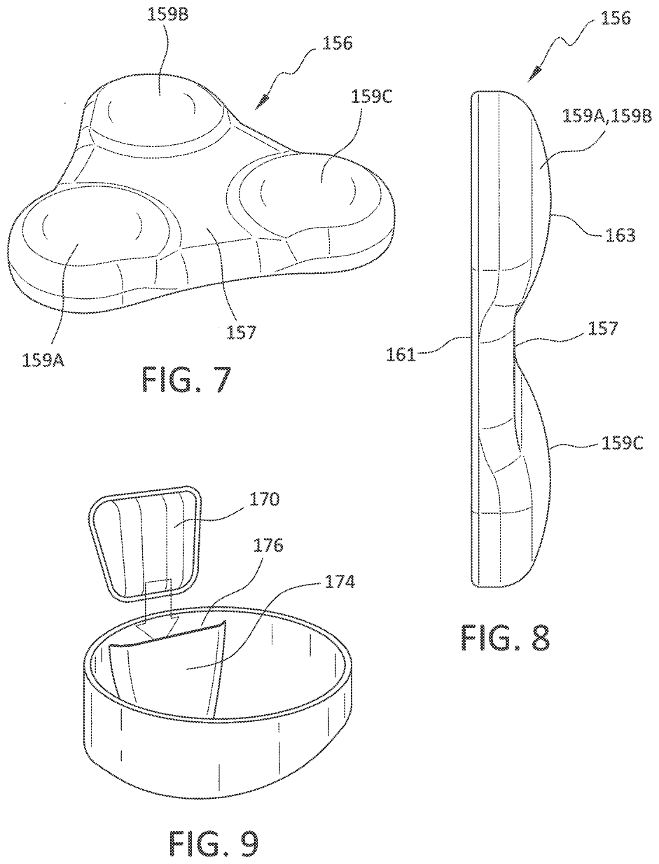

Referring to FIGS. 7-9, a hamstring activator 156 is attached to the sleeve 102. The hamstring activator 156 is arranged for being located on the sleeve 102 and applying pressure corresponding to at least one of the semitendinosus, semimembranosus and biceps femoris tendons. The hamstring activator 156 is arranged to secure to the sleeve 102 at a location proximal of the knee K. The hamstring activator 156 defines a facing surface 157 and a rear surface 161. The hamstring activator 156 has at least one protrusion 159A, 159B, 159C extending from the facing surface 157.

The purpose of the hamstring activator 156 is to target the compression to the hamstring muscles and increase the proprioceptive effect of the orthopedic device on the hamstrings. It is believed that the compression and proprioceptive effect leads to the user being more active and quicker to respond in jumping and cutting movements. As the hamstring stabilizes the knee in the same direction as the ACL, an increase in hamstring activity may make up for deficiency in the ACL.

The hamstring activator 156 has at least two protrusions 159A, 159B, 159C protruding from the facing surface 157. The facing surface 157 is substantially flat relative to the at least one protrusion 159A, 159B, 159C. The at least one protrusion 159A, 159B, 159C has a rounded profile 163. The rear surface 161 is substantially flat and adapted to abut the sleeve 102.

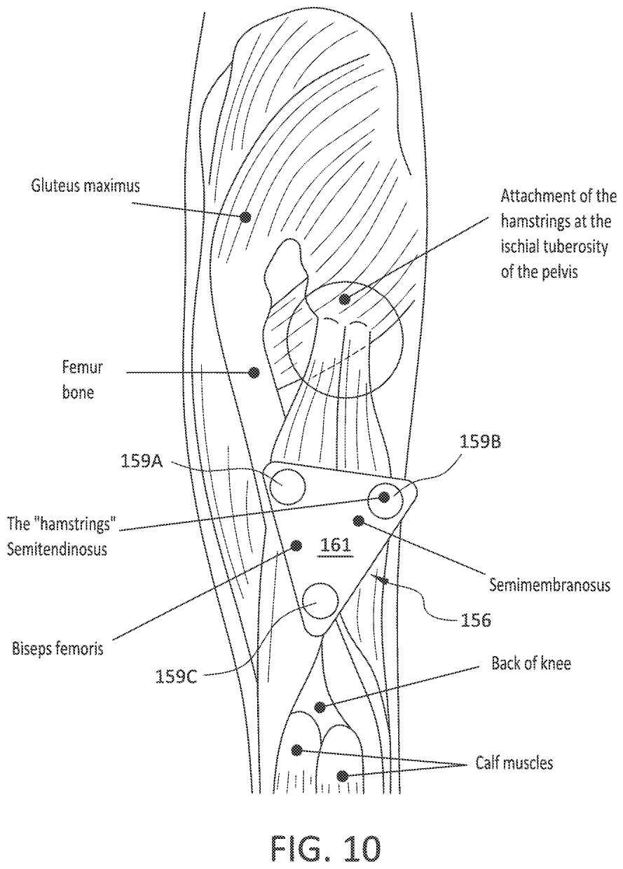

The hamstring activator 156 is flexible and may be formed from a viscoelastic material. The hamstring activator 156 is flexible and resilient and is arranged for the rear surface 161 to approximate a curvature of the sleeve 102. The hamstring activator 156 may be formed from a rigid or semi-rigid material. FIG. 10 shows that the at least one protrusion 159A, 159B, 159C is arranged to apply pressure to at least one of the semitendinosus, semimembranosus and biceps femoris tendons according to inwardly radial elasticity due to the elasticity of the tubular sleeve 102.

The sleeve 102 forms a pocket 176 adapted to receive a hamstring activator 170. The hamstring activator 170 may extend along only a segment of a circumference of the sleeve 102. The pocket 176 may be formed by a flap 174 secured in part to an interior surface of the tubular sleeve 102.

Referring to FIG. 5, the first and second anterior straps 112, 114 are arranged to apply first and second anteriorly directed forces F1, F2 on an anterior leg A, proximal and distal Pr, Di of a knee, respectively. The first and second posterior straps 116, 118 are arranged to apply first and second posteriorly directed forces F3, F4 on a posterior leg P, proximal and distal Pr, Di of a knee, respectively.

A first moment M1 is arranged along a proximal segment of the hinge or stay 108 and a second moment M2 is arranged along a distal segment of the hinge or stay 108 according to tensioning of one of the anterior or posterior straps 112, 114, 116, 118. According to the strap system 103, tensioning of the first and second anterior straps 112, 114 and the first and second posterior straps 116, 118 are arranged to urge a distal end of a femur a distance D9 anteriorly, and a proximal end of a tibia a distance D10 posteriorly according to a stage in gait of a user.

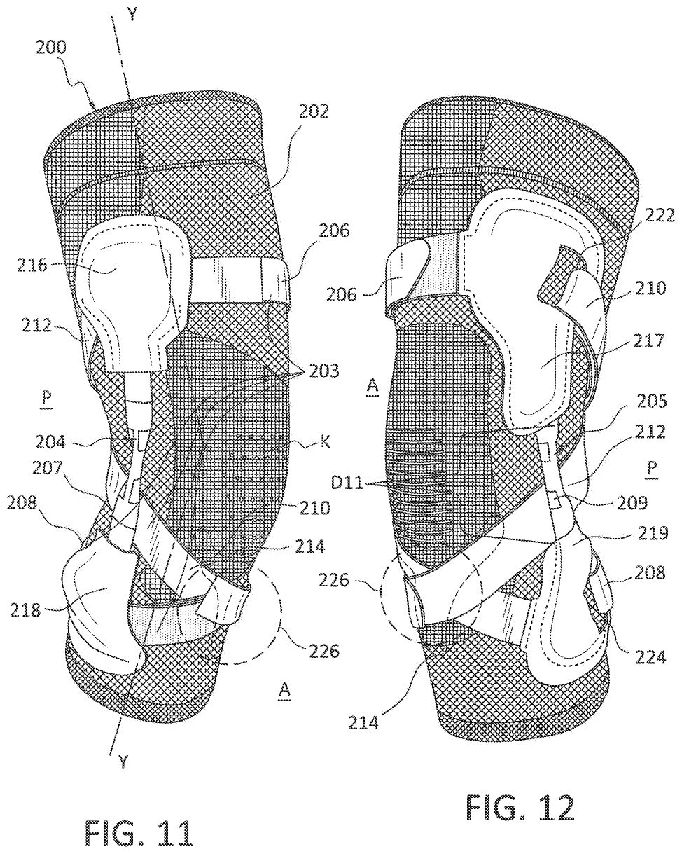

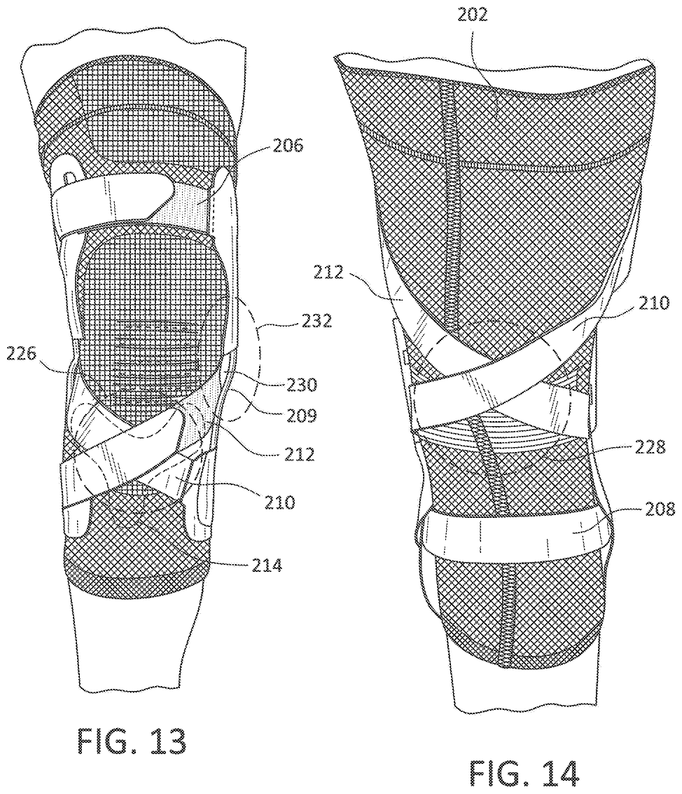

Another embodiment of an orthopedic device 200 is shown in FIGS. 11-14, as another knee support arranged to stabilize a knee or ACL. The knee support 200 includes a sleeve 202 having a tubular form defined along an axis Y-Y. A first hinge 204 extends along a first one of medial or lateral sides of the sleeve 202, and a second hinge 205 extends along a second one of the medial or lateral sides of the sleeve 202. A strapping system 203 may comprise a first anterior strap 206 that has first and second ends connecting to medial and lateral sides of the sleeve 202 on an anterior side A thereof. A first posterior strap 208 of the strapping system 203 has first and second ends connecting to medial and lateral sides of the sleeve 202 on a posterior side P thereof.

A first helical strap 210 connects to the sleeve 202 at a first one of the medial or lateral sides of the sleeve 202 at a proximal side of a knee portion K. The first helical strap 210 extends from the connection at the proximal side of the sleeve 202 along the posterior side P of the sleeve 202 and helically extends about an anterior side A of the sleeve to connect to a second one of the medial or lateral sides of the sleeve 202 at the distal side of the knee portion K. A second helical strap 212 connects to the sleeve 202 at a second one of the medial or lateral sides of the sleeve 202 at a proximal side of a knee portion K. The second helical strap 212 extends from the connection at the proximal side of the sleeve 202 along the posterior side P of the sleeve 202 and helically extends about an anterior side A of the sleeve to connect to the first one of the medial or lateral sides of the sleeve 202 at the distal side of the knee portion K.

The first helical strap 210 extends underneath the first hinge 204 generally near the knee portion K at passing portion 232 along the axis Y-Y of the sleeve 202. Likewise, the second helical strap 212 extends underneath the second hinge 205 generally near the knee portion K at passing portion 232 along the axis Y-Y of the sleeve 202.

The first and second helical straps 210, 212 intersect at posterior intersection 228 along the posterior side P of the sleeve 202 above or at the proximal side Pr proximate or at the knee portion K. The first and second helical straps 210, 212 intersect at an anterior intersection 226 along the anterior side A of the sleeve 202 above or at the distal side Di proximate or at the knee portion K. A pad 214 may be located on the sleeve 202 at the anterior intersection 226.

As with the embodiment of FIGS. 1-4, a first panel section 216, 217 may be provided at a proximal side and a second panel section 218, 219 at a distal side on the medial and lateral sides of the sleeve 202 for receiving ends of one of the first and second hinges 204, 205. A distance D11 of clearance is preferably located between the first and second panel sections 217, 219, and is arranged to expose a middle or articulating segment 207, 209 of the hinge for the first and second helical straps 210, 212 to pass therethrough. The middle or articulating segment 207, 209 of the hinge forms a passageway 230 through which the first and second helical straps 210, 212 pass. First and second slots 222, 224 defined in or by the panel sections may correspond to one or more of the straps and allow passage therethrough for secure engagement.

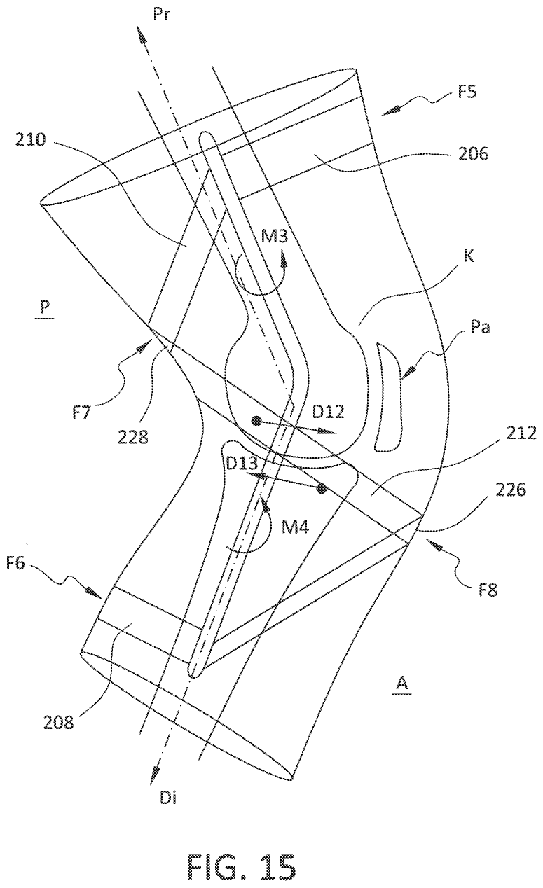

As shown in FIG. 15, the first anterior strap 206 is arranged to apply a first anteriorly directed force F5 on an anterior leg A, proximal Pr of a knee K. The first and second helical straps 210, 212 are arranged to apply a first posteriorly directed force F7 on a posterior leg P at the intersection 228 proximal Pr of a knee. The first and second helical straps 210, 212 are arranged to apply a second anteriorly directed force F8 on an anterior leg A at the intersection 226 distal Di of a knee. The first posterior strap 208 is arranged to apply a second posteriorly directed force F6 on the posterior leg P, distal Di of a knee K. A first moment M1 is arranged along a proximal segment of the hinge or stay 204 and a second moment M2 is arranged along a distal segment of the hinge or stay 108 according to tensioning of one of the anterior or posterior or helical straps 206, 208, 210, 212. Tensioning of the first anterior and posterior straps 206, 208 and the first and second helical straps 210, 212 are arranged to urge a distal end of a femur a distance D12 anteriorly toward a patella Pa, and a proximal end of a tibia a distance D13 posteriorly according a stage in gait of a user.

By providing an orthopedic device for anterior cruciate ligament complications according to the disclosed embodiments, the problems of braces being too rigid and therefore not fostering user compliance, or of not providing sufficiently robust and/or dynamic support from a soft-good brace, are addressed. The orthopedic device for anterior cruciate ligament complications embodiments of the disclosure advantageously provide a sleeve and a strapping system that provide dynamic, staggered support for properly aligning and rotating a tibia relative to a femur of a user while promoting compliance, user comfort, and intuitiveness of use.

While the disclosure discusses embodiments for the knee and ankle, orthopedic device embodiments of the disclosure may be used with other limbs, joints and anatomical portions including the torso, shoulder, elbow, wrist/hand, hip, knee, and foot/ankle.

Not necessarily all such objects or advantages may be achieved under any embodiment of the invention. Those skilled in the art will recognize that the invention may be embodied or carried out to achieve or optimize one advantage or group of advantages as taught without achieving other objects or advantages as taught or suggested.

The skilled artisan will recognize the interchangeability of various components from different embodiments described. Besides the variations described, other known equivalents for each feature can be mixed and matched by one of ordinary skill in this art to construct an orthopedic brace under principles of the present invention. Therefore, the embodiments described may be adapted to orthopedic systems for securing, supporting or comforting limbs or other anatomy.

Although the invention has been disclosed in certain preferred embodiments and examples, it therefore will be understood by those skilled in the art that the present invention extends beyond the disclosed embodiments to other alternative embodiments and/or uses of the invention and obvious modifications and equivalents. It is intended that the scope of the present invention disclosed should not be limited by the disclosed embodiments described above, but should be determined only by a fair reading of the claims that follow.

1. An orthopedic device, comprising:
a sleeve having a tubular form defined along an axis;
a first anterior strap connected to the sleeve and extending between lateral and medial sides of the sleeve on an anterior side thereof;
a second anterior strap connected to the sleeve and extending between the lateral and medial sides of the sleeve on the anterior side;
a first posterior strap connected to the sleeve and extending between the lateral and medial sides of the sleeve on a posterior side thereof;
a second posterior strap connected to the sleeve and extending between the lateral and medial sides of the sleeve on the posterior side.

2. The orthopedic device of claim 1, wherein the first anterior strap and the first posterior strap are located above an anterior center of the sleeve;
wherein the second anterior strap and the second posterior strap are located below the anterior center of the sleeve.

3. The orthopedic device of claim 2, wherein the first posterior strap is located a third distance from the anterior center of the sleeve;
wherein the first anterior strap is located a first distance from the first posterior strap, the first posterior strap is located closer to the anterior center than the first anterior strap.

4. The orthopedic device of claim 2, wherein the second anterior strap is located a fourth distance from the anterior center of the sleeve;
wherein the second posterior strap is located a second distance from the second anterior strap, the second anterior strap is located closer to the anterior center than the second posterior strap.

5. The orthopedic device of claim 1, wherein the first and second anterior straps and the first and second posterior straps are staggered relative to the axis of the sleeve.

6. The orthopedic device of claim 1, wherein the sleeve is formed from an elasticized textile.

7. The orthopedic device of claim 6, further comprising:
a first panel extending along a medial-lateral plane on a first side of the sleeve, the first and second anterior straps and the first and second posterior straps connecting to the first panel;
a first hinge located between the first panel and the sleeve.

8. The orthopedic device of claim 7, wherein the first panel includes a first portion located above the anterior center of the sleeve, a second portion located below the anterior center of the sleeve, and a middle portion located generally at the anterior center of the sleeve;
wherein the first panel further defines a slot through which the first anterior strap extends.

9. The orthopedic device of claim 7, wherein the first and second anterior straps are adjustably secured to the first panel such that adjustment of the first and second anterior straps is in a first circumferential direction, and adjustment of the first and second posterior straps is in a second circumferential direction opposite the first circumferential direction.

10. The orthopedic device of claim 1, further comprising at least one hinge located on at least one of the lateral and medial sides of the sleeve;
wherein the first posterior strap has a first end secured to the first panel;
wherein the first end is defined by an elasticized segment, an inelastic segment of the first posterior strap is secured to the elasticized segment, and adjustably connects to the at least one hinge.

11. The orthopedic device of claim 1, wherein the sleeve defines a popliteal region on the posterior side and has different flexibility from the regions of the sleeve surrounding the popliteal region;
wherein the sleeve defines a knee locator section having a differently formed textile pattern from a periphery about the knee locator section.

12. The orthopedic device of claim 11, wherein the knee locator section defines a flexibility feature defined by a textile pattern providing greater flexibility than the textile pattern about the periphery.

13. The orthopedic device of claim 1, further comprising a hamstring activator attached to the sleeve, the hamstring activator arranged on the sleeve at a region for applying pressure to at least one of the semitendinosus, semimembranosus and biceps femoris tendons.

14. The orthopedic device of claim 13, wherein the hamstring activator defines a facing surface and a rear surface, the hamstring activator having at least one protrusion extending from the facing surface.

15. The orthopedic device of claim 14, wherein the hamstring activator has at least two protrusions protruding from the facing surface;
wherein the facing surface is substantially flat relative to the at least two protrusions;
wherein the rear surface is substantially flat and adapted to abut the sleeve;
wherein the at least two protrusions have a rounded profile.

16. The orthopedic device of claim 14, wherein the at least one protrusion is arranged to apply pressure according to inwardly radial elasticity due to the elasticity of the tubular sleeve.

17. The orthopedic device of claim 13, wherein the hamstring activator is flexible and resilient and is arranged for the rear surface to approximate a curvature of the sleeve.

18. The orthopedic device of claim 13, wherein the sleeve forms a pocket adapted to receive the hamstring activator;
wherein the hamstring activator extends along only a segment of a circumference of the sleeve.

19. An orthopedic device, comprising:
a sleeve having a tubular form defined along an axis;
a first anterior strap connected to the sleeve and extending between lateral and medial sides of the sleeve on an anterior side thereof;
a second anterior strap connected to the sleeve and extending between the lateral and medial sides of the sleeve on a posterior side thereof;
a first posterior strap connected to the sleeve and extending between the lateral and medial sides of the sleeve on the anterior side;
a second posterior strap connected to the sleeve and extending between the lateral and medial sides of the sleeve on the posterior side;
a first hinge located between a first panel and the sleeve;
wherein the first and second anterior straps are arranged to apply first and second anteriorly directed forces on an anterior of a leg, proximal and distal of a knee, respectively;
wherein the first and second posterior straps are arranged to apply first and second posteriorly directed forces on a posterior of a leg, proximal and distal of a knee, respectively;
wherein a first moment is arranged along a proximal segment of the first hinge and a second moment is arranged along a distal segment of the first hinge according to tensioning of one of the anterior or posterior straps.

20. The orthopedic device of claim 19, wherein tensioning of the first and second anterior straps and the first and second posterior straps is arranged to urge a distal end of a femur a distance anteriorly, and a proximal end of a tibia a distance posteriorly according a stage in gait of a user.
